(19) United States
(12) Patent Application Publication
(10) Pub. No.: US 2001/0011147 A1
(43) Pub. Date: Aug. 2, 2001

(54) Method for obtaining components of a packet of additives for engine fuels

(76) Inventors: Andrzej Krueger, Kedzierzyn-Kozle (PL); Boguslaw Tkacz, Kedzierzyn-Kozle (PL); Winicjusz Stanik, Krakow (PL); Leszek Ziemianski, Krakow (PL); Kazimierz Chlobowski, Krakow (PL); Kazimierz Szymanski, Kedzierzyn-Kozle (PL); Ryszard Kosciuk, Kedzierzyn-Kozle (PL); Wladyslaw Millan, Kedzierzyn-Kozle (PL); Anna Rzodeczko, Kedzierzyn-Kozle (PL); Jan Poskrobko, Kedzierzyn-Kozle (PL); Wojciech Balcerowiak, Kedzierzyn-Kozle (PL); Jerzy Jasienkiewicz, Kedzierzyn-Kozle (PL)

Correspondence Address: BAUER & SCHAFFER, 114 Old Country Road, Mineola, NY, 11501, US

(21) Appl. No.: 09/759,082

(22) Filed: Jan. 12, 2001

(30) Foreign Application Priority Data
Jan. 18, 2000 (PL) P.337931

(51) Int. Cl. C07C 43/23

(52) U.S. Cl. 568/593; 568/866; 568/867

(57) ABSTRACT

Method for obtaining components of a packet of additives for engine fuels by oxyalkylenation of organic compounds containing hydroxyl groups by means of alkylene oxide at the presence of basic catalysts, at the temperature 80-170° C., characteristic in that the mixture containing 94.5-99.9% by mass of alkyl phenols of the general formula according to FIG. 1, where R₁-alkyl group of the carbon atoms number from 6 to 16 and not more than 0.1% by mass of water (H₂O) and not more than 5.0% by mass (preferably from 0.1 to 1.0 by mass) of monohydroxyl alcohols of the general formula R₂—OH, where R₂-alkyl group of the carbon atoms number from 1 to 4, is oxyalkylenated with ethylene oxide or propylene oxide up to the moment of obtaining the molecular mass of oxyalkylenated alcohol not lower than 100 daltons and the hydroxyl number not higher than 150 mg of KOH/g, and next the product of synthesis is contacted at the temperature not higher than 150° C. with acid ion-exchange resin in the hydrogen form, favorably with functional sulfo groups, containing at least 0.1 mole of water per 1 mole of functional groups.

Packets of additives used for engine petrols refining, apart from active substances of detergent action, also contain so called carrying oils which support their washing abilities. In the past they were mineral oils, but now depending upon the type of active substance, there are also used synthetic carrying oils which prevent accumulation of sediments and carbon deposits on air-fuel mixture inlet valves in petrol engines. For this purpose it is possible to use among others polyether type chemical compounds.

A method for obtaining this type of a detergent additive for engine fuels was described in the Polish patent no. 175462. Technical nonyl phenol and dodecyl phenol were exposed to oxyalkylenation with propylene oxide or a mixture of propylene oxide and ethylene oxide at the presence of potassium hydroxide as a catalyst, and after demineralisation there was obtained a component of the viscosity from 93 to 118 mPa * s in 40° C. and hydroxyl value from 64 to 74 mg KOH/g, containing the product of oxyalkylenation of nonyl phenol or dodecyl phenol with alkylene oxide in the proportion of 6 to 18 moles of oxide per 1 mole of alkyl phenol. The mole ratio of propylene oxide and ethylene oxide was kept within the range 5:1 to 15:1 and the polyoxyalkylenated chain was formed in the random system.

In the patent literature there are many examples for obtaining and using packets containing components of additives for engine fuels. The U.S. Pat. No. 3,615,295 described obtaining engine petrol with an additive of polyoxyalkylenated alkyl phenols which cause reduction of hydrocarbons content in exhaust gases.

In the Polish patent no. 170 272 there was presented a method for obtaining a packet of additives for engine petrols, which can include alkenylsuccinimides, alkenylsuccinamides, their modified derivatives, obtained according to the description presented in the patent no. P 291 691, synthetic carriers being carbamate or oxyalkylated alkyl phenols as well as substances preventing valve-seats damaging in engines which are not provided with hardened valve-seats. The basic advantage of this version of an additive for engine petrols is high effectiveness of action, increased thermal stability, and introduction of synthetic carriers aids detergent action of active substances. According to the Polish patent no. 172 553 a packet of detergent additives for engine petrols includes derivatives of polypropylene glycol with a substituted —OH functional group. Such a component according to the invention is to improve detergent properties of the detergent additives packet in comparison to the conventional detergent additives for petrols. Polyether type compounds preventing accumulation of carbon and other deposits in the inlet system of air-fuel mixture of spark ignition engines were presented in the patents EP 524783 A1 and EP 549253 A1. The chemical formulas of these compounds are as follows:

RX[(CₓH₂ₓO)ₙ(C_{y}H_{2y}O)ₚ]ₘ or RX[(CₓH₂ₓO)ₙ(C_{y}H_{2y}O)ₚZ]ₘ

Where:

R=alkyl of the carbon atoms number from 7 to 30 or alkyl phenol

X=atom of oxygen, sulphur or nitrogen

Z=hydrocarbon substituent of the carbon atoms number from 1 to 30

x=from 2 to 4, y—from 6 to 30,

m=1 if X-atom of oxygen or sulphur or RX—H, m=2 if X=atom of nitrogen,

n and p fix the ratio of CₓH₂ₓO to C_{y}H_{2y}O

According to the U.S. Pat No. 3,658,494 as synthetic carriers being in packets of additives for engine petrols there are used derivatives of glycol and other polyhydroxyl alcohols, for instance oxy-n-butyl ether of ethylene glycol. According to the Polish patent no. 172 553 polymer derivatives of polypropylene glycol with substituted —OH groups are used together with detergent substances, as more effective synthetic carriers in comparison to the above mentioned ones. Polyether derivatives can be also used as components of packets of additives for diesel fuels, according to the Polish patents 166 515 and 174 112, they are oxyalkylated, preferably oxyethylated and/or oxypropylated, alkyl phenols of the average molecular weight from 100 to 2000 daltons, preferably from 800 to 1500. Detergent additives components for diesel fuels can also be according to the patent JP 04114089 A—derivatives of oxyalkylation of bisphenols and/or their ester derivatives.

In case of synthetic carrier oil special importance is in:

* chemical type of the substance

* physical and chemical properties

* content of by-products which can affect effectiveness of detergent additive action.

Taking into consideration among others a viscosity coefficient, it is favourable to use polymers of higher molecular weights, however, increase of mole ratio of alkylene oxide to alkyl phenol usually leads to higher quantity of low-molecular polyglycols which in the engine work conditions have a tendency to be decomposed forming black deposits. Running the process of oxyalkylenation of organic compounds containing hydroxyl groups with alkylene oxides according to the invention there are obtained polyethers containing derivatives of monohydroxyl alcohols of the average molecular weight 100-200 daltons having corresponding physical and chemical properties and very good usable parameters.

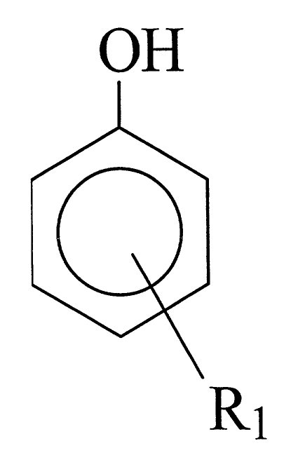

The process according to the invention consists in oxyalkynenation of organic compounds containing hydroxyl groups with alkylene oxides at the temperature 80-170° C. at the presence of basic catalysts. The mixture containing 94.5-99.9% by mass of monoalkyl phenols with an alkyl group of the carbon atoms number from 6 to 16, of the formula according to FIG. 1 and the water content not more than 0.1% by mass, is oxyalkylated with ethylene oxide or propylene oxide at the presence of not more than 5.0% by mass of monohydroxyl alcohols of the general formula R₂—OH, where R₂-alkyl group of the carbon atoms number from 1 to 4, up to the moment of obtaining the molecular weight of oxyalkylenated alcohol not lower than 100 daltons and the hydroxyl number not higher than 150 mg KOH/g. It is favourable to obtain momohydroxyl alcohols content from 0.1% by mass to 1.0% by mass. Next the products of oxyalkylenation are contacted at the temperature of 150° C. with acid ion-exchange resin in the hydrogen form, preferably with functional sulfo groups, containing at least 0.1 mole of water per 1 mole of sulfo groups.

Examples 1 and 2 illustrate a method for obtaining components of a packet of additives for engine fuels according to the invention and Example 3 illustrates the technological state and it is a comparative example.

The advantages of using the invention are improvement of engine fuels quality due to reduction of their tendency to emulsify with water and 2-3 times reduction of sediments and carbon deposits in the inlet system of air-fuel mixture in petrol engines as well as improvement of diesel fuels quality by reduction of their tendency to form carbon deposits on sprayer ends and in consequence to deteriorate and disturb fuel spraying in diesel engines.

### EXAMPLE 1

A pressure reactor of the volume 6 m³ was loaded with 0.6 m³ of corresponding alkyl phenol and added alkaline catalyst in the form of solution in monohydroxyl alcohol, thus there was obtained a mixture for oxyalkylenation of the composition presented in Table 1. Next, the contents of the reactor were heated up to the temperature of 130° C. and dosing of alkylene oxide was started. The process of oxyalkylenation was run at the parameters presented in Table 1 up to obtaining the product containing oxyalkylenated alcohols of the assumed molecular weight. The obtained post-reaction mixture was contacted with strongly acid ion-exchange resin of the type of sulfonated copolymer of styrene with divinylbenzene of controlled water content while maintaining the parameters presented in Table 1.

The product of the characteristics given in Table 2 was used to make a packet of additives for engine fuels.

TABLE 1

| | | | | | | | | Contacting | | | |
|---|---|---|---|---|---|---|---|---|---|---|---|
| Composition of | | | | | | | | cation | | | |
| the mixture for | | | | | Process of | | | exchanger | | | |
| oxyalkylenation | | | | | oxyalkylenation | | | type | | mole | |
| | | Alcohol | | Water | Type of | | | of | | H₂O. | |
| Alkyl | % | | % | % | alkyl. | Cata- | T | cat. | T | mole | Pr. |
| Phenol | b.w | type | b.w. | b.w. | oxide | lyst | [° C.] | ex. | [° C.] | (—SO₃H). | no. |
| hexyl- | 0.3 | | | | | | | | | | |
| phenol | | | | | | | | | | | |
| nonyl- | 0.9 | etha- | 0.3 | 0.05 | pro- | KOH | 140 | gel | 80 | 0.3 | 1 |
| phenol | | nol | | | pylene | | | | | | |
| | | | | | oxide | | | | | | |
| dodecyl | 97.8 | | | | | | | | | | |
| phenol | | | | | | | | | | | |
| hexyl | 0.5 | | | | | | | | | | |
| phenol | | | | | | | | | | | |
| nonyl | 1.2 | meth- | 0.1 | 0.05 | pro- | KOH | 150 | mac- | 80 | 0.3 | 2 |
| phenol | | anol | | | pylene | | | ro- | | | |
| | | | | | oxide | | | por. | | | |
| dodecyl | 96.2 | | | | | | | | | | |
| phenol | | | | | | | | | | | |

TABLE 2

| | Product Characteristics | | |
|---|---|---|---|
| | M. m. of | M. m. of | |
| | oxyalkylenated | oxyalkylenated | Hydroxyl |
| Product | alkyl phenol | alcohol | number |
| Number | (daltons) | (daltons) | mg KOH/g |
| 1 | 890 | 835 | 42 |
| 2 | 1740 | 910 | 37 |

Packet of Additives No.1

Into a mixer provided with an agitator and heating there were added in sequence: 150 kg of the product no.1 of the characteristics given in Table 1, 120 kg of polybuteneamine of the chlorine content below 100 mg/kg and the molecular mass 2100 daltons and 730 kg of naphtha fraction of the ignition temperature 65° C. The components were being mixed for 4 hours at the temperature from 40 to 50° C.

Packet of Additives No.2

Into a mixer provided with an agitator and heating there were added in sequence: 150 kg of the product no. 2 of the characteristics given in Table 1, 120 kg of polybuteneamine of the chlorine content below 100 mg/kg and the molecular mass 2100 daltons and 730 kg of naphtha fraction of the ignition temperature 65° C. The components were being mixed for 4 hours at the temperature from 40 to 50° C. Engine petrol of the characteristics presented in Table 3 was added a packet of additives no.1 or no.2. The composition of the engine petrol was presented in Table 4.

TABLE 3

Engine petrol

| Properties | A | B |
|---|---|---|
| Testing octane number, TON | 95.6 | 94.6 |
| Motor octane number, MON | 84.5 | 87.5 |
| Fraction contents: | | |
| up to 70° C. distilled % (m/m) | 22.1 | 22.5 |
| up to 100° C. distilled % (m/m) | 46.5 | 53.5 |
| up to 180° C. distilled % (m/m) | 93.7 | 94.0 |
| end of distillation, ° C. | 211 | 198 |
| Induction period, min. | >360 | >360 |
| Lead content, g Pb/l | <0.002 | 0.15 |
| Content of methyltert- | 5.9 | — |
| butyl ether % (m/m) | | |
| Density, 20° C., kg/m³ | 760 | 751 |

TABLE 4

| Engine fuel | Type of | Additives packet | Quantity |
|---|---|---|---|
| Number | petrol | number | mg/g |
| 1 | A | 1 | 800 |
| 2 | A | 2 | 800 |
| 3 | A | 1 | 1000 |
| 4 | A | 2 | 1000 |
| 5 | B | 1 | 800 |
| 6 | B | 2 | 800 |
| 7 | B | 1 | 1000 |
| 8 | B | 2 | 1000 |

Engine petrols A and B and engine fuels marked from 1 to 8 underwent engine tests according to the European standard CEC F-04-A-87 making possible estimation of purity of the air-fuel mixture inlet system in the Opel-Kadett engine. The tests results were presented in Table 5.

TABLE 5

| | Test result |
|---|---|
| | [mg carbon deposit and sediment/ |
| Tested petrol | inlet valve] |
| Engine petrol A | 198 |
| Engine fuel no. 1 | 40 |
| Engine fuel no. 2 | 43 |
| Engine fuel no. 3 | 24 |
| Engine fuel no. 4 | 19 |
| Engine petrol B | 233 |
| Engine fuel no. 5 | 25 |
| Engine fuel no. 6 | 27 |
| Engine fuel no. 7 | 15 |
| Engine fuel no. 8 | 8 |

(*) criterion of engine tests requirements meeting: not more than 50 mg of carbon deposit/valve

Engine petrols A and B and engine fuels from 1 to 8 underwent engine tests according to the European standard CEC F-05-A-93 making possible estimation of purity of the air-fuel mixture inlet system in the engine of Mercedes M 102 E. The tests results were presented in Table 6.

TABLE 6

| | Test result *) |
|---|---|
| | [mg carbon deposit and sediment/ |
| Tested petrol | inlet valve] |
| Engine petrol A | 250 |
| Engine fuel no. 1 | 25 |
| Engine fuel no. 2 | 12 |
| Engine fuel no. 3 | 30 |
| Engine fuel no. 4 | 21 |
| Engine petrol B | 415 |
| Engine fuel no. 5 | 35 |
| Engine fuel no. 6 | 17 |
| Engine fuel no. 7 | 40 |
| Engine fuel no. 8 | 23 |

*) criterion of engine tests requirements meeting: not more than 30 mg of carbon deposit/valve for engine petrol according to the World Fuel Chard ACEA.

Engine petrol B and engine fuels marked from 5 to 8 underwent tests for their tendency to emulsify with water. The results were presented in Table 7.

TABLE 7

| | Test result *) | | |
|---|---|---|---|
| | Water layer | Interphase | Distribution |
| | volume | surface | degree |
| Tested petrol | change [ml] | appear. [p.] | [p.] |
| Engine petrol B | 1 | 1 | 1 |
| Engine fuel no. 5 | 1 | 1b | 1 |
| Engine fuel no. 6 | 1 | 2 | 2 |
| Engine fuel no. 7 | 1 | 1b | 1 |
| Engine fuel no. 8 | 1 | 2 | 2 |

*) Permissible level of estimation:
change of water layer volume: not more than 1 ml
interphase surface appearance: estimation max. 2 points
distribution degree: estimation max. 2 points.

### EXAMPLE 2

Into a vacuum reactor of the volume 6 m³ there were loaded: 2 m³ of corresponding alkyl phenol and a alkaline catalyst in the form of monohydroxyl alcohol, thus obtaining a mixture for oxyalkylenation of the composition given in Table 8. Next, the contents of the reactor were heated up to the temperature of 130° C. and alkylene oxide dosing was started. The process of oxyalkylenation was run at the parameters presented in Table 8 up to obtaining the product containing oxyalkylenated alcohols of the assumed molecular weight. The obtained post-reaction mixture was contacted with strongly acid ion exchange resin of the type of sulfonated copolymer of styrene with divinyl benzene of the controlled water content, while maintaining the parameters given in Table 8.

TABLE 8

| | | | | | | | | Contacting | | | |
|---|---|---|---|---|---|---|---|---|---|---|---|
| Composition of | | | | | | | | cation | | | |
| the mixture for | | | | | Process of | | | exchanger | | | |
| oxyalkylenation | | | | | oxyalkylenation | | | type | | mole | |
| | | Alcohol | | Water | Type of | | | of | | H₂O | |
| Alkyl | % | | % | % | alkyl. | Cata- | T | cat. | T | mole | Pr. |
| phenol | b.w. | type | b.w. | b.w. | oxide | lyst | [° C.] | ex. | [° C.] | (—SO₃H). | no. |
| hexyl- | 0.3 | | | | | | | | | | |
| phenol | | | | | | | | | | | |
| nonyl- | 95.1 | meth- | 5.0 | 0.1 | ethy- | NaOH | 100 | gel | 120 | 0.1 | 3 |
| phenol | | anol | | | lene | | | | | | |
| | | | | | oxide | | | | | | |
| do- | 0.1 | | | | | | | | | | |
| decyl- | | | | | | | | | | | |
| phenol | | | | | | | | | | | |
| hexyl- | 0.2 | | | | | | | | | | |
| phenol | | | | | | | | | | | |
| nonyl- | 0.5 | iso- | 1.0 | 0.07 | ethy- | NaOH | 120 | mac- | 100 | 0.1 | 4 |
| phenol | | pro- | | | lene | | | ro- | | | |
| | | panol | | | oxide | | | por | | | |
| do- | 99.0 | | | | | | | | | | |
| decyl- | | | | | | | | | | | |
| phenol | | | | | | | | | | | |

The product of the characteristics presented in Table 9 was used to make a packet of additives for engine fuels.

TABLE 9

| | Product characteristics | | |
|---|---|---|---|
| | m.m. of | m.m. of | |
| | oxyalkylated | oxyalkylated | hydroxy |
| Product | alkyl phenol | alcohol | number |
| Number | (daltons) | (daltons) | mg of KOH |
| 3 | 400 | 130 | 147 |
| 4 | 530 | 150 | 102 |

Packet of Additives No. 3

Into a mixer provided with an agitator and heating there were added in sequence: 100 kg of the product no. 3 of the characteristics given in Table 9, 100 kg of alkenyl-succinimide of the average molecular mass 2350 daltons, 100 kg of Mannich base of the average molecular mass 580 daltons and 200 kg of naphtha fraction of the ignition temperature 65° C. The components were being mixed for 4 hours at the temperature from 40 to 50° C.

Packet of Additives No. 4

Into a mixer provided with an agitator and heating there were added in sequence: 150 kg of the product no.4 of the characteristics given in Table 9, 100 kg of alkenyl-succinimide of the average molecular mass 2350 daltons, molecular mass 2100 daltons, 100 kg of Mannich base of the average molecular mass 580 daltons and 200 kg of naphtha fraction of the ignition temperature 65° C. The components were being mixed for 4 hours at the temperature from 40 to 50° C.

Diesel fuel of the characteristics given in Table 10 was added a packet of additives no.3 or no.4. The composition of the engine fuel was presented in Table 11.

TABLE 10

| Diesel Fuel Properties | Diesel Fuel A | Diesel Fuel B |
|---|---|---|
| Cetane number | 50.9 | 49.0 |
| Cetane index | 52.5 | 50.4 |
| Fraction composition, ° C. | | |
| boiling start | 184 | 175 |
| 10% distillates | 213 | 207 |
| 50% distillates | 263 | 269 |
| 90% distillates | 328 | 350 |
| distillation end | 354 | 378 |
| Post-coking residue, % (m/m) | 0.012 | 0.078 |
| Sulphur content, % (m/m) | 0.048 | 0.042 |
| Density, 20° C. g/cm³ | 0.831 | 0.837 |

TABLE 11

| Engine Fuel | Diesel Fuel | Additives Packet | Quantity, |
|---|---|---|---|
| Number | Type | Number | mg/kg |
| 9 | A | 3 | 500 |
| 10 | A | 4 | 500 |
| 11 | B | 3 | 500 |
| 12 | B | 4 | 500 |

Diesel fuels A and B of the characteristics given in Table 10 and engine fuels obtained on the basis of them, marked from 9 to 12, underwent engine testing according to the procedure CEC PF 26 in an engine of Peugeot XUD 9. The test estimates a tendency of diesel fuel to form carbon deposits at the sprayers ends, and thus to deteriorate and disturb fuel spraying. The criterion of estimation is reduction of air flow through a sprayer end after the test is over in comparison to its flow before it is started. The World-Wide Fuel Charter of December 1998 accepts max. 85% reduction of air flow through a sprayer end with needle lifting for 0.1 mm. The engine tests results were presented in Table 12.

TABLE 12

| Diesel Fuel | Air Flow Change (%) |
|---|---|
| Diesel fuel A | 87 |
| Engine fuel no. 9 | 65 |
| Engine fuel no. 10 | 62 |
| Diesel fuel B | 91 |
| Engine fuel no. 11 | 75 |
| Engine fuel no. 12 | 65 |

### EXAMPLE 3

Into a mixer provided with an agitator and heating there were added in sequence: 150 kg of the product obtained according to the method of the patent PL 175 462 of the average molecular weight 783 daltons and the hydroxyl group 78 mg KOH/g, 120 kg of polybutene-amine of chlorine content below 100 mg/kg and the molecular mass 2100 daltons and 730 kg of naphtha fraction of the ignition temperature 65° C. The composition was being mixed for 4 hours at the temperature from 40 to 50° C. The packet of additives thus obtained was marked no.5. Engine petrol of the characteristics presented in Table 3 was added the packet of additives no. 1 or no. 2. Composition of obtained engine fuel was presented in Table 13.

TABLE 13

| Engine Fuel | Petrol | Additives packet | |
|---|---|---|---|
| Number | Type | Number | Quantity, mg/kg |
| 13 | A | 5 | 800 |
| 14 | A | 5 | 1000 |
| 15 | B | 5 | 800 |
| 16 | B | 5 | 1000 |

Engine fuels marked from 13 to 16 underwent engine testing according to the European standard CEC F-04-A-87 making possible estimation of purity of the fuel-air mix inlet system in an engine of Opel-Kadett. The test results were presented in Table 14.

TABLE 14

| | Test Result*⁾ |
|---|---|
| | mg of carbon deposits and sediments/ |
| Tested Petrol | / inlet valve |
| Engine fuel no. 13 | 85 |
| Engine fuel no. 14 | 62 |
| Engine fuel no. 15 | 72 |
| Engine fuel no. 16 | 47 |

^{(*)} Criterion of engine test requirements fulfilling: not more than 50 mg of carbon deposits/valve.

Engine fuels of the examples from 13 to 16 underwent engine tests according to the European standard CEC F-05-A-93 making possible estimation of purity of the fuel-air mix inlet system in an engine of Mercedes M 102 E. The tests results were presented in Table 15.

TABLE 15

| | Test Result *) |
|---|---|
| | mg of carbon deposits and sediments |
| Tested Petrol | /inlet valve |
| Engine fuel no. 13 | 79 |
| Engine fuel no. 14 | 55 |
| Engine fuel no. 15 | 72 |
| Engine fuel no. 16 | 41 |

(*) Criterion of engine test requirements fulfilling: not more than 30 mg of carbon deposits/valve for engine petrol according to the World Fuel Charter ACEA.

1. Method for obtaining components of a packet of additives for engine fuels by oxyalkylenation of organic compounds containing hydroxyl groups by means of alkylene oxide at the presence of basic catalysts, at the temperature 80-170° C., characteristic in that the mixture containing 94.5-99.9% by mass of alkyl phenols of the general formula according to FIG. 1, where R₁-alkyl group of the carbon atoms number from 6 to 16 and not more than 0.1% by mass of water (H₂O) and not more than 5.0% by mass (preferably from 0.1 to 1.0 by mass) of monohydroxyl alcohols of the general formula R₂—OH, where R₂-alkyl group of the carbon atoms number from 1 to 4, is oxyalkylenated with ethylene oxide or propylene oxide up to the moment of obtaining the molecular mass of oxyalkylenated alcohol not lower than 100 daltons and the hydroxyl number not higher than 150 mg of KOH/g, and next the product of synthesis is contacted at the temperature not higher than 150° C. with acid ion-exchange resin in the hydrogen form, favourably with functional sulfo groups, containing at least 0.1 mole of water per 1 mole of functional groups.
